(19) United States
(12) Patent Application Publication
(10) Pub. No.: US 2023/0026340 A1
(43) Pub. Date: Jan. 26, 2023

(54) METHODS AND COMPOSITIONS FOR PROMOTING PLANT GROWTH AND ROOT NODULATION

(71) Applicant: BASF Corporation, Florham Park, NJ (US)

(72) Inventors: Jessica M. STONE, Tarrytown (US); Janine REIMANN, Tarrytown (US); Piran CARGEEG, Saskatoon (CA)

(21) Appl. No.: 17/780,412

(22) PCT Filed: Nov. 25, 2020
(86) PCT No.: PCT/US2020/062411

§ 371 Date: May 26, 2022

Related U.S. Application Data
(60) Provisional application No. 62/941,225, filed on Nov. 27, 2019.

(51) Int. Cl. A01N 63/20; A01P 21/00

(52) U.S. Cl. CPC A01N 63/20; A01P 21/00

(57) ABSTRACT

The disclosure provides for methods for increasing plant vigor and root nodule development comprising applying to seeds at least one Methylobacterium and at least one rhizobium. The disclosure further provides for compositions comprising at least one Methylobacterium and at least one rhizobium. Compounds, compositions, seeds, and plants useful in these methods and obtained therefrom are also described.

### FIELD

The disclosure provides for methods for increasing plant vigor and root nodule development comprising applying to seeds, in-furrow and/or soil at least one Methylobacterium and at least one rhizobium (including members of Rhizobiaceae, Bradyrhizobiaceae and Phylllobacteriaceae Families). The disclosure further provides for compositions comprising at least one Methylobacterium and at least one rhizobium.

Compounds, compositions, seeds, applications and plants useful in these methods and obtained therefrom are also described.

### BACKGROUND

Root nodules are found on the roots of plants (notably legumes) which form a symbiosis with nitrogen-fixing bacteria. Under nitrogen-limiting conditions, capable plants form a symbiotic relationship with a host-specific species of bacteria belonging to multiple taxonomic families, herewith defined as “rhizobium”. This process has evolved multiple times within legumes, as well as other selected plant species found within the Rosid clade.

Most of this nitrogen is provided through biological fixation of atmospheric nitrogen by endophytic bacteria living in colonies or “nodules” within the plant roots. Inside the legume root nodules, di-nitrogen gas from the atmosphere (N₂) is converted primarily into ammonium (NH4+), which is subsequently assimilated into amino acids, nucleotides, and other cellular constituents such as vitamins, flavones, and hormones. Legumes' ability to fix gaseous nitrogen makes them an ideal agricultural organism as their requirement for mineral/fertilizer nitrogen is reduced. High nitrogen content in soil can inhibit nodule development as there is no benefit for the plant of forming the symbiosis. The energy for splitting the nitrogen gas in the nodule comes from carbohydrates (primarily sucrose) translocated from the leaf. Malate as a break-down product of sucrose is used as the direct carbon source for the bacteroid.

Nitrogen fixation in the nodule is oxygen sensitive. Legume nodules produce iron and molybdenum cofactors which enable “fixation” of atmospheric nitrogen byway of a microaerophilic oxygen creating phytoglobin-leghaemoglobin, closely related to haemoglobin found in mammals.

Although the vast majority of plants able to form nitrogen-fixing root nodules are in the legume family Fabaceae, there are a few exceptions, such as Parasponia, a tropical genus in the Cannabaceae family, which is also able to interact with rhizobia and form nitrogen-fixing nodules.

Soybean plants have a high demand for nitrogen; approximately five pounds of nitrogen are typically required to produce one bushel of harvested soybean. Biological fixation from the atmosphere accounts for est. 50-75 percent of a soybean crop's total nitrogen requirement, with the remainder being acquired from the soil.

Because biological fixation can be a significant source of fixed nitrogen for soybean plants, soybean farmers evaluate soybean plants in each field to assess whether nodules are present in sufficient numbers and whether they are actively fixing nitrogen. Nodules normally appear about 6 weeks after planting and should be actively fixing nitrogen at this time. Nodules which actively fix atmospheric nitrogen are pink or red when cut open (indicating leghaemoglobin presence). If nodules are very small and white, they may be immature and have not begun fixing nitrogen. Nodules which are otherwise white, green, brown or black will not fix nitrogen.

Poor nodulation and reduced nitrogen fixation are most likely to occur in non-inoculated, new non-rotated soybean fields due to low bacteria populations in the soil; in fields containing high levels of residual soil nitrogen, in coarse-textured soils due to inadequate moisture levels to sustain bacteria, in flooded or saturated soil conditions lasting seven days or more due to oxygen deprivation, in soil having pH below approx. 5.0 or above approx. 8.5, and/or in soils which are compacted due to reduced oxygen availability.

Fields showing nitrogen deficiency symptoms (chlorosis and stunted plants) due to low nodule numbers or inactive nodules may respond to supplemental nitrogen. Applying nitrogen fertilizer prior to full bloom can provide an economic return in certain situations. However, when more fertilizer is added at one time than plants can uptake and retain, the excess fertilizer ends up in groundwater.

Because soybeans require large amounts of nitrogen and biological fixation is a major source of nitrogen, maximization of biological nitrogen fixation may ultimately achieve higher yields.

As soybeans are one of the most versatile and commercially valuable crops around the world, there is a need to develop new methodology and compositions which improve the overall efficiency and production of soybean plants to meet global demand while also minimizing environmental impact. In particular, there is a need to develop new methods and compositions which achieve enhanced nitrogen fixation, including earlier nodulation and increase in number of effective root nodules and total nodule biomass per plant.

### SUMMARY OF VARIOUS EMBODIMENTS

The disclosure provides for methods for increasing plant vigor and root nodule development comprising applying to seeds, plants, plant parts, soil, and/or in-furrow at least one Methylobacterium sp. and at least one rhizobium.

The disclosure further provides for compositions comprising at least one Methylobacterium sp. and at least one rhizobium.

In an aspect, the disclosure provides for methods for improving plant health and/or yield potential comprising applying to a seed, plant, soil/in-furrow and/or part or habitat thereof a composition comprising
a) one or more Methylobacterium sp.; and
b) one or more genus/species of rhizobium.

In an aspect, the disclosure provides for methods for improving plant health and/or yield potential comprising applying to a seed, plant, soil/in-furrow and/or part or habitat thereof
a) a first composition comprising one or more Methylobacterium sp.; and
b) a second composition comprising one or more genus/species of rhizobium.

In an aspect, the disclosure provides for methods for enhancing plant vigor comprising applying to a seed, plant, soil/in-furrow and/or part or habitat thereof a composition comprising
a) one or more Methylobacterium sp.; and
b) one or more genus/species of rhizobium.

In an aspect, the disclosure provides for methods for enhancing plant vigor comprising applying to a seed, plant, soil, in-furrow and/or part or habitat thereof
a) a first composition comprising one or more Methylobacterium sp.; and
b) a second composition comprising one or more genus/species of rhizobium.

In another aspect, the disclosure provides for compositions for improving plant health and/or yield potential comprising:
a) one or more Methylobacterium sp.; and
b) one or more genus/species of rhizobium.

In another aspect, the disclosure provides for use of a composition for improving plant health and/or yield potential comprising
a) one or more Methylobacterium sp.; and
b) one or more genus/species of rhizobium for improving plant health and/or yield potential.

### BRIEF DESCRIPTION OF THE DRAWINGS

FIG. 1 depicts the number of nodules observed on soybean plants grown from seeds according to the present invention after 15 days, 18 days, 21 days, and 28 days after inoculation, wherein the seeds were treated with B. japonicum strain 532c at 2*10⁴ cfu per seed and with varying concentrations of Methylobacterium radiotolerans.

FIG. 2 depicts the number of nodules observed on soybean plants grown from seeds according to the present invention after 15 days, 18 days, 21 days, and 28 days after inoculation, wherein the seeds were treated with B. japonicum strain 532c at 2*10⁵ cfu per seed and with varying concentrations of Methylobacterium radiotolerans.

FIG. 3 depicts the accumulated average number of nodules and average dry weight per nodule measured on a total of ten soybean plants grown from seeds according to the present invention three weeks after inoculation, measured against a negative control, a positive control of SEMIA 5080, and a positive control of Methylobacterium radiotolerans.

FIG. 4 depicts the accumulated average number of nodules measured on a total of ten soybean plants grown from seeds according to the present invention six weeks after inoculation, measured against a negative control, a positive control of SEMIA 5080, and a positive control of 532c.

FIG. 5 depicts the average dry weight per nodule measured on a total of ten soybean plants grown from seeds according to the present invention six weeks after inoculation.

FIG. 6 depicts nodules obtained from ten soybean plants grown from seeds according to the present six weeks after inoculation: 532c control (top left), SEMIA 5080 control (top right), 532c+M. radiotolerans (bottom left), and SEMIA 5080+radiotolerans (bottom right).

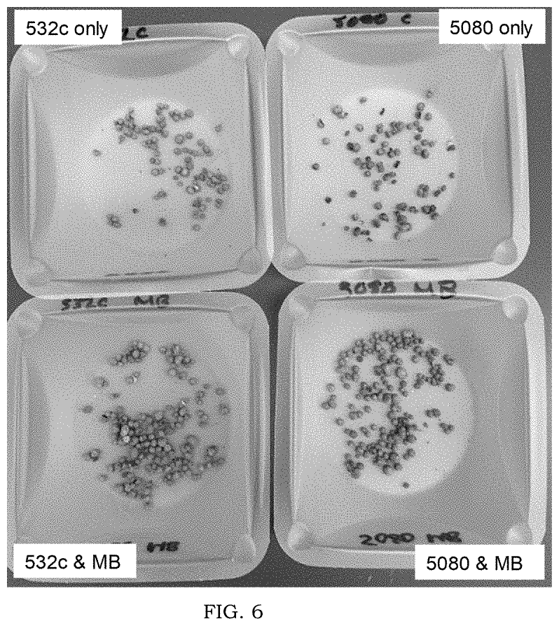

### DETAILED DESCRIPTION OF VARIOUS EMBODIMENTS

The disclosure provides methods for increasing plant vigor and root nodule development comprising applying to seeds and/or in-furrow to the soil at least one Methylobacterium sp. and at least one rhizobium. The disclosure further provides for compositions comprising at least one Methylobacterium sp. and at least one rhizobium.

Compounds, compositions, seeds, applications and plants useful in these methods and obtained therefrom are also described.

In an aspect, compositions according to the present invention comprise one or more species belonging to the genus Methylobacterium.

In an aspect, compositions according to the present invention comprise one or more families/genera/species of rhizobium. In an aspect, any strain of any species of rhizobium may be used.

In certain embodiments, compositions according to the present invention comprise one or more species belonging to the genus Methylobacterium and one or more families/genera/species of rhizobium.

In an aspect, compositions according to the present invention may comprise any species of Methylobacterium.

In certain embodiments, the species of Methylobacterium is selected from, but not limited to, the group consisting of M. aminovorans, M. extorquens, M. fujisawaense, M. mesophilicum, M. radiotolerans, M. rhodesianum, M. nodulans, M. phyllosphaerae, M. thiocyanatum, and M. oryzae.

In certain embodiments, the Methylobacterium is selected from the group consisting of M. aminovorans, M. extorquens, M. filfisawaense, M. mesophilicum, M. radiotolerans, M. rhodesianum, M. nodulans, M. phyllosphaerae, M. thiocyanatum, and M. oryzae. In certain embodiments, the Methylobacterium species is selected from isolate deposited with the Agricultural Research Service Culture Collection (NRRL) of the National Center for Agricultural Utilization Research (Agricultural Research Service, U.S. Department of Agriculture, 1815 North University Street, Peoria, Ill. 61604) at deposit numbers NRRL B-50929, NRRL B-50930, NRRL B-50931, NRRL B-50932, NRRL B50933, NRRL B-50934, NRRL B-50935, NRRL B-50936, NRRL B-50937, NRRL B-50938, NRRL B-50939, NRRL B-50940, NRRL B-50941, NRRL B-50942 and derivatives thereof.

In certain embodiments, the Methylobacterium species is not M. nodulans.

In certain embodiments, the Methylobacterium species is not M. aminovorans, M. extorquens, M filjisawaense, M mesophilicum, M. rhodesianum, M nodulans, M phyllosphaerae, M. thiocyanatum, or M. oryzae.

In certain embodiments, the Methylobacterium species is Methylobacterium radiotolerans, strain DSM1819, deposited with International Deposit Authority ATCC at deposit number ATCC 27329, CIP 101128, IAM 12098 (full scientific name: Methylobacterium radiotolerans (Ito and Iizuka 1971) Green and Bousfield 1983) (see bacdive.dsmz.de/strain/7152 and Ito, H., Iizuka, H. (1971). Taxonomic studies on a radio-resistant Pseudomonas. Part XII. Studies on the microorganisms of cereal grain. Agric. Biol. Chem. 35: 1566-1571).

In certain embodiments, the families/genus/species/strain of nitrogen fixing rhizobium are selected from, but not limited to, the group consisting of one or more Azospirillum extracts (e.g., an extract of media comprising A. brasilense INTA Az-39), one or more Bradyrhizobium extracts (e.g., an extract of media comprising B. elkanii SEMIA 501, B. elkanii SEMIA 587, B. elkanii SEMIA 5019, B. japonicum NRRL B-50586 (also deposited as NRRL B-59565), B. japonicum NRRL B-50587 (also deposited as NRRL B59566), B. japonicum NRRL B-50588 (also deposited as NRRL B-59567), B. japonicum NRRL B-50589 (also deposited as NRRL B-59568), B. japonicum NRRL B-50590 (also deposited as NRRL B-59569), B. japonicum NRRL B-50591 (also deposited as NRRL B59570), B. japonicum NRRL B-50592 (also deposited as NRRL B-59571), B. japonicum NRRL B-50593 (also deposited as NRRL B-59572), B. japonicum NRRL B-50594 (also deposited as NRRL B-50493), B. japonicum NRRL B-50608, B. japonicum NRRL B-50609, B. japonicum NRRL B-50610, B. japonicum NRRL B-50611, B. japonicum NRRL B-50612, B. japonicum NRRL B-50726, B. japonicum NRRL B-50727, B. japonicum NRRL B-50728, B. japonicum NRRL B-50729, B. japonicum NRRL B-50730, B. japonicum SEMIA 566, B. japonicum SEMIA 5079, B. japonicum SEMIA 5080, B. japonicum USDA 6, B. japonicum USDA 110, B. japonicum USDA 122, B. japonicum USDA 123, B. japonicum USDA 127, B. japonicum USDA 129 and/or B. japonicum USDA 532C), one or more Rhizobium extracts (e.g., an extract of media comprising R. leguminosarum S012A-2), one or more Sinorhizobium extracts (e.g., an extract of media comprising S. fredii CCBAU114 and/or S. fredii USDA 205), one or more Penicillium extracts (e.g., an extract of media comprising P. bilaiae ATCC 18309, P. bilaiae ATCC 20851, P. bilaiae ATCC 22348, P. bilaiae NRRL 50162, P. bilaiae NRRL 50169, P. bilaiae NRRL 50776, P. bilaiae NRRL 50777, P. bilaiae NRRL 50778, P. bilaiae NRRL 50777, P. bilaiae NRRL 50778, P. bilaiae NRRL 50779, P. bilaiae NRRL 50780, P. bilaiae NRRL 50781, P. bilaiae NRRL 50782, P. bilaiae NRRL 50783, P. bilaiae NRRL 50784, P. bilaiae NRRL 50785, P. bilaiae NRRL 50786, P. bilaiae NRRL 50787, P. bilaiae NRRL 50788, P. bilaiae RS7B-SD1, P. brevicompactum AgRF18, P. canescens ATCC 10419, P. expansum ATCC 24692, P. expansum YT02, P. fellatanum ATCC 48694, P. gaestrivorus NRRL 50170, P. glabrum DAOM 239074, P. glabrum CBS 229.28, P. janthmellum ATCC 10455, P. lanosocoeruleum ATCC 48919, P. radicum ATCC 201836, P. radicum FRR 4717, P. radicum FRR 4719, P. radicum N93/47267 and/or P. raistrickii ATCC 10490), one or more Pseudomonas extracts (e.g., an extract of media comprising P. jessenii PS06), Yersinia entomophaga 082KB8 and combinations thereof.

In certain embodiments, the families/genus/species of nitrogen fixing rhizobium are selected from, but not limited to, the group consisting of Bradyrhizobium japonicum, Bradyrhizobium diazoefficiens, B. elkanii, Bradyrhizobium spp., Bradyrhizobium sp. (Arachis), Bradyrhizobium sp. (Vigna), B. liaoningense, B. lupine; Mesorhizobium spp., Mesorhizobium ciceri, M huakii, M. loti; Rhizobium leguminosarum bv. phaseoli, R. leguminosarum bv. trifolii, R. leguminosarum bv. viciae, R. tropici, and Sinorhizobium meliloti.

In certain embodiments, the species of Methylobacterium is M. radiotolerans.

In certain embodiments, the genus/species/strain of rhizobium is Bradyrhizobium diazoefficiens SEMIA 5080, Bradyrhizobium japonicum 532c, Rhizobium leguminosarum bv. viceae and Bradyrhizobium sp. (Arachis).

In an aspect, the composition comprising one or more species of Methylobacterium is applied to seed, in-furrow, to the soil, a plant or part and/or habitat thereof at a rate of 0.10 to 10⁹ cfu/seed, or 1 to 10⁸ cfu/seed, or 10 to 10⁷ cfu/seed, or 100 to 5*10⁶ cfu/seed.

In an aspect, the composition comprising one or more species of Methylobacterium is applied to seed, in-furrow, to the soil, a plant or part and/or habitat thereof at a rate of greater than 10 cfu/seed, or at a rate of greater than 100 cfu/seed, or at a rate of greater than 10³ cfu/seed.

In an aspect, the composition comprising one or more species of Methylobacterium is applied to soil at a rate of 10⁶ to 10¹⁶ cfu/hectare, or 10⁷ to 10¹⁵ cfu/hectare, 10⁸ to 10¹⁴ cfu/hectare, or 10⁹ to 10¹³ cfu/hectare, or 10¹⁰ to 10¹² cfu/hectare.

In an aspect, the composition comprising one or more family/genus/species of rhizobium is applied to seed, in-furrow, to the soil, a plant or part and/or habitat thereof at a rate of 0.10 to 10⁹ cfu/seed, or 1 to 10⁸ cfu/seed, or 10 to 10⁷ cfu/seed, or 100 to 5*10⁶ cfu/seed.

In an aspect, the composition comprising one or more family/genus/species of rhizobium is applied to seed, in-furrow, to the soil, a plant or part and/or habitat thereof at a rate of greater than 10 cfu/seed, or at a rate of greater than 100 cfu/seed, or at a rate of greater than 10³ cfu/seed.

In an aspect, the composition comprising one or more family/genus/species of rhizobium is applied to soil at a rate of 10⁶ to 10¹⁶ cfu/hectare, or 10⁷ to 10¹⁵ cfu/hectare, 10⁸ to 10¹⁴ cfu/hectare, or 10⁹ to 10¹³ cfu/hectare, or 10¹⁰ to 10¹² cfu/hectare.

In an aspect, a composition according to the present invention comprises one or more species/strains of Methylobacterium at a concentration of 0.01 to 10¹⁶ cfu/mL, or 0.1 to 10¹⁵ cfu/mL, or 1 to 10¹⁴ cfu/mL, or 10 to 10¹⁴ cfu/mL, or 10² to 10¹³ cfu/mL.

In an aspect, a composition according to the present invention comprises one or more species/strains of Methylobacterium at a concentration of 0.01 to 10⁶ cfu/mL.

In an aspect, a composition according to the present invention comprises one or more species/strains of Methylobacterium at a concentration of 10⁸ cfu/mL to 10¹⁶ cfu/mL.

In an aspect, a composition according to the present invention comprises one or more species/strains of Methylobacterium at a concentration of 0.01 to 10¹⁶ cfu/g, or 0.1 to 10¹⁵ cfu/g, or 1 to 10¹⁴ cfu/g, or 10 to 10¹⁴ cfu/g, or 10² to 10¹³ cfu/g.

In an aspect, a composition according to the present invention comprises one or more family/genus/species/strains of rhizobium at a concentration of 0.01 to 10¹⁶ cfu/mL, or 0.1 to 10¹⁵ cfu/mL, or 1 to 10¹⁴ cfu/mL, or 10 to 10¹⁴ cfu/mL, or 10² to 10¹³ cfu/mL.

In an aspect, a composition according to the present invention comprises one or more family/genus/species/strains of rhizobium at a concentration of 0.01 to 10⁶ cfu/mL.

In an aspect, a composition according to the present invention comprises one or more family/genus/species/strains of rhizobium at a concentration of 10⁸ cfu/mL to 10¹⁶ cfu/mL.

In an aspect, a composition according to the present invention comprises one or more family/genus/species/strains of rhizobium at a concentration of 0.01 to 10¹⁶ cfu/g, or 0.1 to 10¹⁵ cfu/g, or 1 to 10¹⁴ cfu/g, or 10 to 10¹⁴ cfu/g, or 10² to 10¹³ cfu/g.

In an aspect, soybean plants grown according to the present invention exhibit increased nodule mass as measured in dry weight of nodules as compared to soybean plants that were not grown according to the present invention.

In an aspect, soybean plants grown according to the present invention exhibit an average dry weight per nodule which is 1% to 250% greater, or 5% to 200% greater, or 10% to 175% greater, or 25% to 150% greater than the average dry weight of nodules of soybean plants that were not grown according to the present invention.

In an aspect, soybean plants grown according to the present invention exhibit a total dry weight of nodules which is 1% to 250% greater, or 5% to 200% greater, or 10% to 175% greater, or 25% to 150% greater than the total dry weight of nodules of soybean plants that were not grown according to the present invention.

In an aspect, soybean plants grown according to the present invention possess nodules in greater quantity than soybean plans that were not grown according to the present invention. In an aspect, soybean plants grown according to the present invention possess about 10% to about 500% more nodules, about 20% to about 400% more nodules, about 35% to about 300% more nodules, about 50% to about 200% more nodules, about 20% to about 100% more nodules, about 25% to about 75% more nodules than soybean plants not grown according to the present invention.

The present inventors have surprisingly found that treatment of seeds with Methylobacterium and rhizobium results in an unexpected, complementary effect on plant growth and root nodule formation.

In an aspect, compositions according to the present invention exhibit a complementary effect on plant growth and root nodule formation.

In certain embodiments, one or more compositions according to the present invention is/are applied to seed in a pre-planting step.

In other embodiments, one or more compositions according to the present invention is/are applied to seed, in-furrow, soil and/or a habitat in which seed has been planted in a post-planting step.

In certain embodiments, one or more compositions according to the present invention is/are applied to seed in a pre-germination application step.

In certain embodiments, one or more compositions according to the present invention is/are applied to seed or a plant growing or grown therefrom in a post-germination application step.

In certain embodiments, one or more compositions each comprising one or more species/strains of Methylobacterium is/are applied to seed, in-furrow and/or soil followed by application of one or more compositions each comprising one or more species of rhizobia.

In certain embodiments, one or more compositions each comprising one or more families/genera/species/strains of rhizobium is/are applied to seed, in-furrow and/or soil followed by application of one or more compositions each comprising one or more species/strains of Methylobacterium.

In certain embodiments, one or more composition comprising one or more species/strains Single step of Methylobacterium and rhizobium are applied simultaneously or as a combined tank mix.

In an aspect, methods and compositions according to the present invention may be applied to any plant or crop or any seed or part thereof, or in-furrow, soils and/or to any habitat in which any plant or crop is growing or may be grown.

In an aspect, any plant or part or seed thereof may be treated according to the present invention.

In certain embodiments, the crop or plant is any legume.

In certain embodiments, the crop or plant is a soybean plant.

In an aspect, any method of coating or otherwise inoculating seed may be employed.

In certain embodiments, the compositions and methods of the present invention employ on-seed coating.

In certain embodiments, the compositions and methods of the present invention employ in-furrow and/or soil application.

The disclosure also provides for plants and plant parts that are coated or partially coated with a composition comprising a one or more Methylobacterium and one or more rhizobium. In certain embodiments, the plant or plant part is coated or partially coated with a composition comprising one or more Methylobacterium and one or more rhizobium.

In certain embodiments, compositions according to the present invention may comprise a solid substance having one or more Methylobacterium grown thereon and adhered thereto and/or one or more rhizobium grown thereon and adhered thereto.

In other embodiments, the compositions according to the present invention may comprise a co-formulation having one or more Methylobacterium grown or added therein and/or one or more rhizobia grown or added therein. In an aspect, a composition according to the present invention is applied to any plant part. In certain embodiments, the plant part is a seed, a stem, a flower, a leaf, a petiole, a pod, and an axillary bud.

In an aspect, a composition according to the present invention is applied in-furrow or to the soil prior to or at planting or seeding.

As used herein, “adhered thereto” or ““adherent” denotes Methylobacterium or rhizobia grown within and/or associated with a solid substance by growing or having been grown on a solid substance.

In an aspect, any agriculturally acceptable adjuvant may be incorporated into compositions according to the present invention. As used herein, the phrase “agriculturally acceptable adjuvant” denotes a substance that enhances the performance of an active agent in a composition for treatment of plants and/or plant parts. In certain embodiments, an active agent may comprise a mono-culture or co-culture of Methylobacterium and/or a mono-culture or co-culture of rhizobium.

In an aspect, any agriculturally acceptable excipient may be incorporated into compositions according to the present invention. As used herein, the phrase “agriculturally acceptable excipient” denotes an essentially inert substance that can be used as a diluent and/or carrier for an active agent in a composition for treatment of seeds, plants and/or plant parts in-furrow and/or soil. In certain embodiments, an active agent can comprise a monoculture or co-culture of Methylobacterium and/or a mono-culture or co-culture of rhizobium.

As used herein, “co-culture of Methylobacterium” refers to a Methylobacterium culture comprising at least two strains of Methylobacterium and/or at least two species of Methylobacterium.

As used herein, “co-culture of rhizobia” refers to a rhizobium culture comprising at least two strains of rhizobium and/or at least two members taken from the Rhizobiaceae, Bradyrhizobiaceae and Phyllobacteriaceae families.

As used herein, the term “Methylobacterium” denotes any bacteria that are facultative methylotrophs of the genus Methylobacterium.

In an aspect, compositions according to the present invention may optionally further comprise one or more pest control and or biostimulant (sometimes referred to as supplements, natural plant stimulants, plant growth promoters etc.) agents, such as, but not limited to, insecticides, fungicides, miticides, acaricides, selective herbicides, biologicals, pheromones, biochemicals and semiochemicals.

In an aspect, the methods according to the present invention may include applying at least one optional pest control agent, such as an insect control agent or a fungal control agent, to a seed, plant, or plant part. Optionally, the insect or fungal control agent, if present, may be applied to soil in which a seed is to be planted according to the method of the present invention, or may be applied to a planted seed or soil in which seed is already planted according to the present invention. Alternatively, the insect or fungal control agent, if present, may be mixed with a composition according to the present invention, and applied simultaneously with the spore forming bacterium. Optionally, the insect or fungus control agent can be applied separately to the seed, plant, or plant part.

If the bacteria-containing composition(s) of the present invention and optional insect or fungal control agent are in powder form, they may be applied directly to the soil, seed, or foliar separately or mixed together at the time of use. If in liquid form, the bacteria-containing composition(s) bacterium and insect or fungal control agent may be sprayed or atomized foliarly or in-furrow at the time of planting, either separately or mixed together at the time of treating. Alternatively, the liquid combination can be introduced to the soil before germination of the seed or directly to the soil in contact with the roots by utilizing a variety of techniques included, but not limited to, drip irrigation, sprinklers, soil injection or soil drenching. In certain embodiments, the liquid is applied to the seed before planting.

Depending on the final formulation and method of application, one or more suitable additives can also be introduced to compositions of the present invention. Additives such as carboxymethylcellulose and natural and synthetic polymers in the form of powders, granules, or latexes, such as gum Arabic, chitin, polyvinyl alcohol and polyvinyl acetate, as well as natural phospholipids, such as cephalins and lecithins, and synthetic phospholipids, can be added to the present compositions.

Optionally, stabilizers and buffers can be added, including alkaline and alkaline earth metal salts and organic acids, such as citric acid and ascorbic acid, inorganic acids, such as hydrochloric acid or sulfuring acid. Biocides can also be added and can included formaldehydes or formaldehyde-releasing agents and derivatives of benzoic acid, such as p-hydroxybenzoic acid. Further additives include functional agents capable of protecting seeds from harmful effects of selective herbicides such as activated carbon, nutrients (fertilizers), and other agents capable of improving the germination and quality of the products or a combination thereof.

In an aspect, the spore-forming bacterium and optional insect control agents are formulated as a liquid seed treatment. The seed treatment comprises at least one spore forming bacterium, and at least one optional insect control agent.

Optionally, a fungicide control agent can be mixed with the spore-forming bacterium and insect control agent. The seeds are substantially uniformly coated with one or more layers of spore-forming bacterium, optional insect control agent, and optional fungicide control agent, using conventional methods of mixing, spraying or a combination thereof. Application is done using specifically designed and manufactured equipment that accurately, safely, and efficiently applies seed treatment products to seeds. Such equipment uses various types of coating technology such as rotary coaters, drum coaters, fluidized bed techniques, spouted beds, rotary mists or a combination thereof. Preferably, the application is done via either a spinning “atomizer” disk or a spray nozzle which evenly distributes the seed treatment onto the seed as it moves through the spray pattern. Preferably, the seed is then mixed or tumbled for an additional period of time to achieve additional treatment distribution and drying. The seeds can be primed or unprimed before coating with the inventive compositions to increase the uniformity of germination and emergence. In an alternative embodiment, a dry powder composition can be metered onto the moving seed.

In an aspect, the seeds according to the present invention may be coated via a continuous or batch coating process. In a continuous coating process, continuous flow equipment simultaneously meters both the seed flow and the seed treatment products. A slide gate, cone and orifice, seed wheel, or weight device (belt or diverter) regulates seed flow. Once the seed flow rate through treating equipment is determined, the flow rate of the seed treatment is calibrated to the seed flow rate in order to deliver the desired dose to the seed as it flows through the seed treating equipment. Additionally, a computer system may monitor the seed input to the coating machine, thereby maintaining a constant flow of the appropriate amount of seed. In a batch coating process, batch treating equipment weighs out a prescribed amount of seed and places the seed into a closed treating chamber or bowl where the corresponding of seed treatment is then applied. The seed and seed treatment are then mixed to achieve a substantially uniform coating on each seed. This batch is then dumped out of the treating chamber in preparation for the treatment of the next batch.

With computer control systems, this batch process is automated enabling it to continuously repeat the batch treating process. In either coating process, the seed coating machinery can optionally be operated by a programmable logic controller that allows various equipment to be started and stopped without employee intervention. The components of this system are commercially available through several sources such as Gustafson Equipment of Shakopee, Minn.

In an aspect, a variety of additives can be added to the seed treatments. Binders can be added and include those composed preferably of an adhesive polymer that can be natural or synthetic without phytotoxic effect on the seed to be coated. A variety of colorants may be employed, including organic chromophores classified as nitroso, nitro, azo, including monoazo, bisazo, and polyazo, diphenylmethane, triarylmethane, xanthene, methane, acridine, thiazole, thiazine, indamine, indophenol, azine, oxazine, anthraquinone, and phthalocyanine. Other additives that can be added include trace nutrients such as salts of iron, manganese, boron, copper, cobalt, molybdenum, and zinc. A polymer or other dust control agent can be applied to retain the treatment on the seed surface.

Other conventional seed treatment additives include, but are not limited to, coating agents, wetting agents, buffering agents, and polysaccharides. At least one agriculturally acceptable carrier can be added to the seed treatment formulation such as water, solids or dry powders. The dry powders can be derived from a variety of materials such as wood barks, calcium carbonate, gypsum, vermiculite, talc, humus, activated charcoal, and various phosphorous compounds.

In one embodiment, the seed coating can comprise of at least one filler, which is an organic or inorganic, natural or synthetic component with which the active components are combined to facilitate its application onto the seed. In certain embodiments, the filler is an inert solid such as clays, natural or synthetic silicates, silica, resins, waxes, solid fertilizers (for example ammonium salts), natural soil minerals, such as kaolins, clays, talc, lime, quartz, attapulgite, montmorillonite, bentonite, or diatomaceous earths, or synthetic minerals, such as silica, alumina, or silicates, in particular aluminum or magnesium silicates.

In an aspect, a compound or composition described herein is formulated as a foliar composition, a foliar spray, solutions, emulsions, suspension, coating formulation, non-pesticidal or pesticidal coating formulation, encapsulated formulation, solid, liquid, fertilizer, paste, granule, powder, suspension, or suspension concentrate. In another aspect, a compound or composition described herein may be employed alone or in solid, dispersant, or liquid formulation. In yet another aspect, a compound or composition described herein is formulated as a tank-mix product. In another aspect, a compound or composition described herein is formulated as a soil application formulation, such as a drench formulation or granular spray formulation.

The following examples serve to illustrate certain aspects of the disclosure and are not intended to limit the disclosure.

### EXAMPLES

### Example 1—Methylobacterium radiotolerans

The present inventors determined that Methylobacterium radiotolerans has plant-promoting characteristics and therefore was tested in conjunction with SEMIA 5080 and in conjunction with 532c to determine if there was a positive effect on nodulation and plant growth.

Methylobacterium radiotolerans grows with a pink pigmentation, which was determined through analytics to be associated with carotenoid production. Due to the UV protectant nature of carotenoids, and the observations that the Methylobacterium and SEMIA 5080 were growing closely together on plates, this bacterium was tested to see if there were any potential “complementary” plant performance benefits. A plant study was conducted coating soybean seeds with both SEMIA 5080 and Methylobacterium radiotolerans.

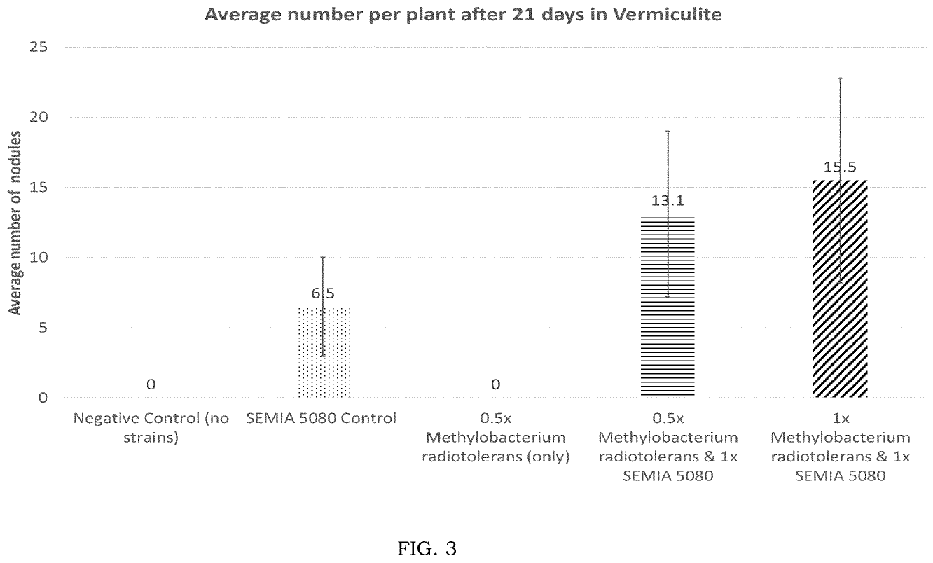

See FIG. 3, which depicts nodule number average between seeds coated with (i) 0.5× Methylobacterium radiotolerans and (ii) 1× SEMIA 5080, 1:1 ratio of the two and finally (iii) one coated only with SEMIA 5080. There was a 138% increase in the number of nodules formed for the seeds coated with a 1:1 ratio of Methylobacterium radiotolerans and SEMIA 5080 and a 101% increase for the seeds coated with a 0.5:1 ratio compared to the SEMIA 5080 control.

As shown in FIG. 3, the seeds coated with both SEMIA 5080 and Methylobacterium radiotolerans, show to have an unexpectedly positive, complementary effect on the production of nodules. These results show that Methylobacterium radiotolerans can be used to enhance SEMIA 5080 activity and nodulation performance.

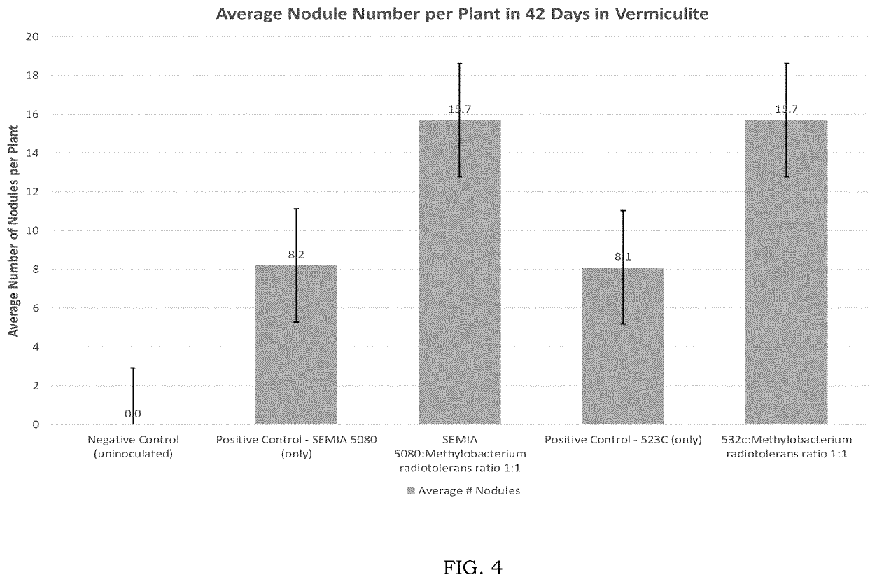

FIG. 4 depicts an additional growth study with co-coated soybean seeds that were grown for 6 weeks. As depicted in FIG. 4, there is a 91% increase in number of nodules formed for seeds coated with a combination of Methylobacterium radiotolerans and SEMIA 5080 compared to the SEMIA 5080 control. There is a 94% increase in number of nodules formed for seeds coated with a combination of Methylobacterium radiotolerans and 532c compared to the 532c control. T test p-values were calculated. Co-coated seeds with SEMIA 5080 have a p-value of 0.004 and seeds co-coated with 532c have a p-value of 0.000.

A repeat study was conducted to show if under nitrogen limitation (6 weeks) the increase in nodule number repeats with SEMIA 5080 and 532c. Results are consistent, showing a significant increase in nodule production when seeds are coated with a combination of Bradyrhizobium and Methylobacterium radiotolerans.

The effect seen with SEMIA 5080 is also consistent for 532c. Thus, the unexpectedly complementary effect was repeatable, and the co-coating effect is similar for a secondary Bradyrhizobium species.

### Example 2—Raw Growth Data

Table 1 sets forth raw growth data:

TABLE 1

| | | | Dry Weight |
|---|---|---|---|
| | Average # | Dry Weight of | per Nodule |
| Total Averages | Nodules | All Nodules (mg) | (mg/Nodule) |
| Negative Control | 0.0 | 0.0 | 0.0 |
| 5080 Positive Control | 8.2 | 145.0 | 17.7 |
| SEMIA 5080/ | 15.7 | 229.3 | 16.2 |
| M. radiotolerans | | | |
| 532c Positive Control | 8.1 | 131.2 | 14.6 |
| 532c/M. radiotolerans | 15.7 | 220.1 | 14.0 |

### Example 3—M. radiotolerans with Bradyrhizobium

In an initial study, the present inventors concluded that Methylobacterium radiotolerans alone does not support soybean nodulation.

When planted in combination with Bradyrhizobium diazoefficiens SEMIA 5080, however, a large increase in the number of nodules was surprisingly observed at 3 weeks.

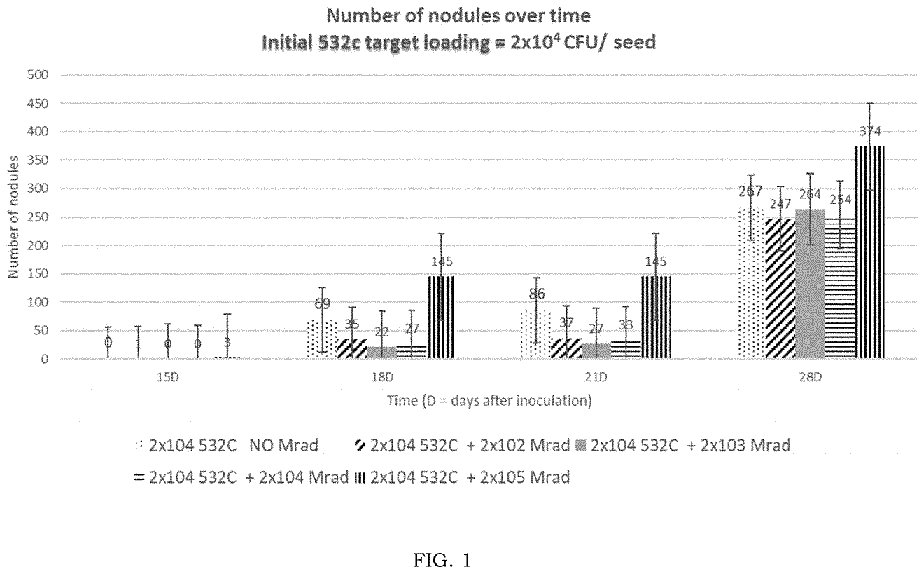

Early nodulation would not be expected with application of 532c alone. As depicted in FIG. 1, early nodulation was not observed in the 2e4 cfu/seed 532c positive control. After 18 days, only 69 nodules were present in the positive control, whereas 145 were present when the 2e4 cfu/seed 532c+2e5 cfu/seed Methylobacterium radiotolerans combination was applied.

Initial plant growth test combining the Methylobacterium radiotolerans and SEMIA 5080 showed promising results for increased, and earlier nodule production.

FIG. 3 depicts the substantial increase in number of nodules after only three weeks, in particular a 101% increase using a 0.5× M. radiotolerans: 1× SEMIA 5080 inoculation, as compared to a positive SEMIA 5080 control.

Furthermore, FIG. 3 depicts a similar substantial increase in the number of nodules after only three weeks, in particular a 93% increase using a 1× M. radiotolerans: 1× SEMIA 5080 inoculation, as compared to a positive SEMIA 5080 control.

A secondary plant study with SEMIA 5080 and 532c strains showed a statistically significant increase in the average number of nodules formed compared to the controls. FIG. 4 depicts the substantial increase in number of nodules after six weeks, in particular a 91% increase when using a SEMIA 5080/M. radiotolerans inoculation as compared to a positive SEMIA 5080 control.

Likewise, FIG. 4 shows a substantial increase of 94% in nodule count when using a 532c/M. radiotolerans inoculation as compared to a positive 532c control.

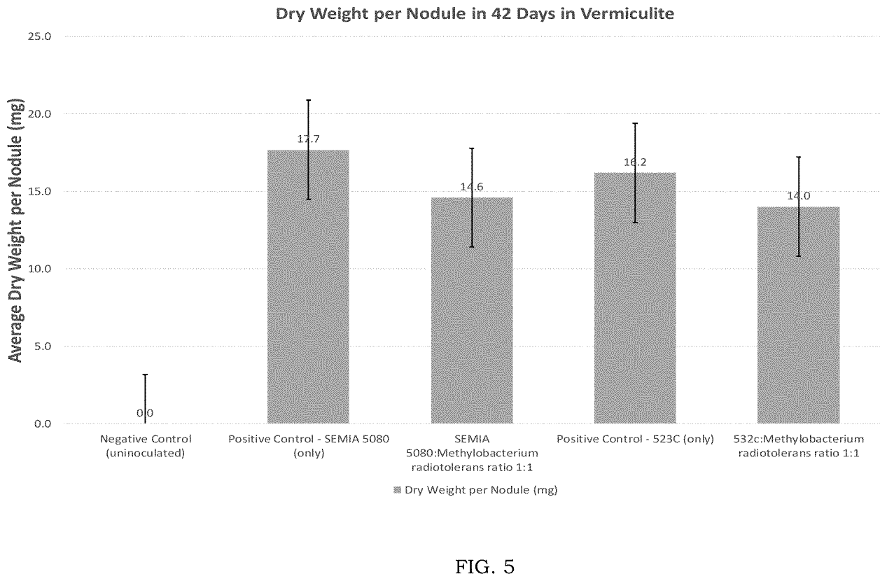

Furthermore, FIG. 5 depicts a decrease in average dry weight per nodule. A 17.4% decrease in average dry nodule weight was measured in soybean plants when using a SEMIA 5080/M. radiotolerans inoculation as compared to a positive SEMIA 5080 control.

Likewise, a 13.4% decrease in average dry nodule weight was measured in soybean plants when using a 532c/M. radiotolerans inoculation as compared to a positive 532c control.

FIG. 5 provides further data demonstrating the unexpected complementary effect achieved by combining M. radiotolerans with either rhizobium strain of SEMIA 5080 or 532c.

### Example 4—Initial 532c Target Loading at 2*10⁴ cfu/Seed and 2*10⁵ cfu/Seed

A nodulation study was conducted comparing effects on various application rates of M. radiotolerans to soybean with an initial 532c target loading of 2*10⁴ cfu/seed.

B. japonicum 532c was cultured in Yeast Mannitol based growth media while M. radiotolerans was cultured in selective growth media.

1 mL of each component was added per seed onto four (4) pre-germinated soybean seeds. Nodule number per plant and per pouch-replicate were recorded 15, 18, 21 and 28 days after inoculation. For each application rate indicated, there were ten (10) replicates.

FIG. 1 depicts the results of applying no M. radiotolerans (positive 532c control), M radiotolerans at 2*10² cfu/seed plus 532c at 2*10⁴ cfu/seed, M radiotolerans at 2*10³ cfu/seed plus 532c at 2*10⁴ cfu/seed, M. radiotolerans at 2*10⁴ cfu/seed plus 532c at 2*10⁴ cfu/seed, and M. radiotolerans at 2*10⁵ cfu/seed plus 532c at 2*10⁴ cfu/seed.

A further nodulation study was conducted comparing effects on various application rates of M. radiotolerans to soybean with an initial 532c target loading of 2*10⁵ cfu/seed.

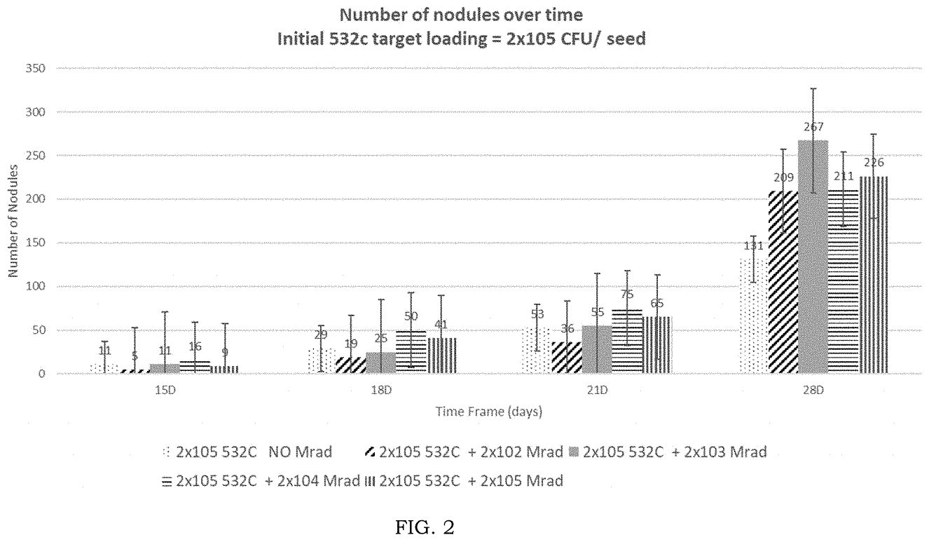

FIG. 2 depicts the results of applying no M. radiotolerans (positive 532c control), M. radiotolerans at 2*10² cfu/seed plus 532c at 2*10⁵ cfu/seed, M radiotolerans at 2*10³ cfu/seed plus 532c at 2*10⁵ cfu/seed, M. radiotolerans at 2*10⁴ cfu/seed plus 532c at 2*10⁵ cfu/seed, and M. radiotolerans at 2*10⁵ cfu/seed plus 532c at 2*10⁵ cfu/seed.

In both studies, substantial increases in the number of nodules were observed in combinations comprising M. radiotolerans. When 532c was initially loaded at 2*10⁴ cfu/seed (see FIG. 1), application of M. radiotolerans at 2*10⁵ cfu/seed produced an obvious complementary benefit.

Moreover, when 532 was initially loaded at 2*10⁵ cfu/seed (see FIG. 2), application of M. radiotolerans at any application rate clearly resulted in nodulation benefits.

The results of these additional nodulation studies suggest that M. radiotolerans has positive, complementary impact on 532C nodulation. Earlier nodulation and increased nodulation will positively impact nitrogen fixation, plant health and quantitative and qualitative plant yield potential.

The data indicates a possibility of increased nodulation with reduced (2e4 cfu/seed) rhizobial loading in combination with M. radiotolerans at 2e5 cfu/seed target loading.

Furthermore, on-seed survivability could be extended, indicated by a potential to reduce the threshold requirement from 1e5 to 1e4 cfu/seed.

1. A method for improving plant nodulation, plant health and/or yield potential comprising applying to a legume seed or plant or part, in-furrow, soil, or habitat thereof one or more compositions comprising
a) one or more Methylobacterium sp.; and
b) one or more rhizobium selected from the families Rhiobiaceae, Bradyrhiobiaceae and Ph llobacteriaceae.

2. The method according to claim 1, wherein the one or more species of rhizobium strain is SEMIA 5080 and/or 532c.

3. The method according to claim 1, wherein the rhizobium is SEMIA 5080.

4. The method according to claim 1, wherein the rhizobium is 532c.

5. The method according to claim 1, wherein the Methylobacterium is M. radiotolerans.

6. The method according to claim 1, wherein the plant is a soybean plant.

7. The method according to claim 1, wherein total dry root nodule weight is increased relative to plants grown from seeds to which the composition has not been applied.

8. The method according to claim 1, wherein total dry root nodule weight is increased 1% to 250% relative to plants grown from seeds to which the composition has not been applied.

9. The method according to claim 1, wherein the total number of root nodules present is increased relative to plants grown from seeds to which the composition has not been applied.

10. The method according to claim 1, wherein the total number of root nodules present is increased from about 10% to about 200% relative to plants grown from seeds to which the composition has not been applied.

11. The method according to claim 1, wherein the one or more species of rhizobium is applied at a rate of about 0.10 to about 10⁹ cfu/seed.

12. The method according to claim 1, wherein the one or more Methylobacterium sp. is applied at a rate of about 0.10 to about 10⁹ cfu/seed.

13. A composition for improving plant nodulation, plant health and/or yield potential comprising:
a) one or more Methylobacterium sp.; and
b) one or more species of rhizobium.

14. The composition according to claim 13, wherein the one or more species of rhizobium is SEMIA 5080 and/or 532c.

15. The composition according to claim 13, wherein the rhizobium is SEMIA 5080.

16. The composition according to claim 13, wherein the rhizobium is 532c.

17. The composition according to claim 13, wherein the Methylobacterium is M. radiotolerans.

18. The method according to claim 1, wherein the one or more Methylobacterium present in the composition is applied to seed at a rate of 0.10 to 10⁹ cfu/seed, or 1 to 10⁸ cfu/seed, or 10 to 10⁷ cfu/seed, or 100 to 5×10⁶ cfu seed.

19. The method according to claim 1, wherein the one or more Methylobacterium present in the composition is applied to soil at a rate of 10⁶ to 10¹⁶ cfu/hectare, or 10⁷ to 10¹⁵ cfu/hectare, 10⁸ to 10¹⁴ cfu/hectare, or 10⁹ to 10¹³ cfu/hectare, or 10¹⁰ to 10¹² cfu/hectare.

20. The method according to claim 1, wherein the one or more species of rhizobium present in the composition is applied to seed at a rate of 0.10 to 10⁹ cfu/seed, or 1 to 10⁸ cfu/seed, or 10 to 10⁷ cfu/seed, or 100 to 5×10⁶ cfu seed.

21. The method according to claim 1, wherein the one or more species of rhiozbium present in the composition is applied to soil at a rate of 10⁶ to 10¹⁶ cfu/hectare, or 10⁷ to 10¹⁵ cfu/hectare, 10⁸ to 10¹⁴ cfu/hectare, or 10⁹ to 10¹³ cfu/hectare, or 10¹⁰ to 10¹² cfu/hectare.

22. The method according to claim 20, wherein the one or more rhizobium present in the composition is SEMIA 5080 and/or 532c.

23. (canceled)
